(19) United States
(12) Patent Application Publication
(10) Pub. No.: US 2024/0132631 A1
(43) Pub. Date: Apr. 25, 2024

(54) METHOD OF PREPARING GANODERMA LUCIDUM BETA-GLUCAN AND A DETECTION METHOD THEREFOR

(71) Applicants: Yinjun HOU, Xi'an (CN); Hua Li, Xi'an (CN); Qian Gao, Xi'an (CN); Yongbo Wang, Xi'an (CN); Xiuding Yang, Xi'an (CN); Yue Gao, Xi'an (CN); Yuqing Zhao, Xi'an (CN); Jie Zhang, Xi'an (CN); Chengyuan Liang, Xi'an (CN)

(72) Inventors: Yinjun HOU, Xi'an (CN); Hua Li, Xi'an (CN); Qian Gao, Xi'an (CN); Yongbo Wang, Xi'an (CN); Xiuding Yang, Xi'an (CN); Yue Gao, Xi'an (CN); Yuqing Zhao, Xi'an (CN); Jie Zhang, Xi'an (CN); Chengyuan Liang, Xi'an (CN)

(73) Assignee: SHAANXI ANGSHENG BIO-PHARMACEUTICAL TECHNOLOGY CO., LTD., Xi'an (CN)

(21) Appl. No.: 18/163,103

(22) Filed: Feb. 1, 2023

(30) Foreign Application Priority Data
Oct. 18, 2022 (CN) 202211281114.9

(51) Int. Cl. C08B 37/00; C12P 19/04

(52) U.S. Cl. CPC C08B 37/0024; C08B 37/0003; C12P 19/04

(57) ABSTRACT

The invention provides a preparation method of high-content Ganoderma lucidum β-glucan and an efficient and rapid detection method thereof. The preparation method comprises ethanol-trypsin pretreatment of Ganoderma lucidum fruiting body in combination with ultrasonic-assisted alkaline hydrothermal extraction of β-glucan from Ganoderma lucidum, and purifying β-glucan by DEAE Sephadex A-25 Sephadex column chromatography using NAOH solution as a mobile phase, to obtain high purity (≥90%) of Ganoderma lucidum β-glucan. The detection method comprises placing the extracted β-glucan in an autoclave and performing hydrolysis with β-(1,3)-exo-glucanase and β-glucosidase, and detecting and analyzing the content of the prepared β-glucan by high pressure-acid enzymatic hydrolysis-HPLC method, using glucose as a standard. The preparation method of β-glucan has high extraction ratio. The efficient and rapid detection method has high sensitivity, high accuracy and good repeatability, and thus has a wide application prospect.

This application claims priority to Chinese Patent Application No. 202211281114.9, filed on Oct. 19, 2022, which is incorporated by reference for all purposes as if fully set forth herein.

### FIELD OF THE INVENTION

The present application relates to the field of medicine, and specifically adopts alcohol-pancreatin pretreatment combined with ultrasonic-assisted alkaline hydrothermal extraction of β-glucan in Ganoderma lucidum, purifies with DEAE Sephadex A-25 glucan gel column chromatography, and utilizes high pressure-Acidase hydrolysis-HPLC method to determine the content of the prepared β-glucan.

### DESCRIPTION OF THE RELATED ART

Ganoderma (Ganoderma lucidum (Curtis) P. Karst.), with an umbrella-shaped shape and a kidney-shaped, semi-circular or nearly circular cap, is the whole plant of Ganoderma lucidum, a Polyporaceae plant. Since ancient times, Ganoderma has been a precious Chinese medicinal material. It was originally wild and mainly grown in Zhejiang, Guangxi, Jiangxi, Hunan and other places. Now with the improvement of human cultivation technology, it is mainly cultivated, and Shandong has the largest output. With the continuous deepening of research, more biological activities of Ganoderma lucidum have been discovered, mainly used for liver protection and detoxification, treatment of diabetes, improvement of cardiovascular system, prevention of aging, and improvement of immunity.

Ganoderma lucidum polysaccharide is one of the most effective components in Ganoderma lucidum. It is extracted from Ganoderma lucidum spores or Ganoderma lucidum fruiting bodies. At present, there are more than 200 kinds of Ganoderma lucidum polysaccharides, most of which are β-glucan, and a few are α-glucan. β-glucan is the most active component of Ganoderma lucidum polysaccharides. It plays an important role in clinical practice due to its immunomodulatory, anti-tumor, anti-colitis, antibacterial, antiviral and other activities. Therefore, it is of great medical significance to study the separation method and quantitative detection and analysis.

At present, the methods mainly used for preparing Ganoderma β-glucan include hot water extraction, ultrasonic extraction, microwave extraction, etc. The extraction efficiency is low, the time is long, and the product contains many impurities. There is an urgent need for a high-content Ganoderma β-glucan. Preparation method of glucan. The invention establishes a method for preparing high-content Ganoderma lucidum β-glucan by ethanol-pancreatin pretreatment combined with ultrasonic-assisted alkaline hydrothermal extraction. The method is simple, the product has high purity and the extraction efficiency is high. Using glucose as the standard substance, a high pressure-acid enzymatic hydrolysis-HPLC method was established to detect the prepared β-glucan, which is simple, rapid, sensitive and accurate, and has broad application prospects.

### SUMMARY OF THE INVENTION

An object of the present invention is to provide a preparation method of high-content Ganoderma lucidum β-glucan and an efficient and rapid detection method thereof. The β-glucan in Ganoderma lucidum is prepared by ethanol-pancreatin pretreatment combined with ultrasonic-assisted alkaline hydrothermal extraction, and the β-glucan content is quantitatively detected and analyzed by a high pressure-acid enzymatic hydrolysis-HPLC method, which provides an effective and reliable analytical method for the preparation and determination of Ganoderma lucidum β-glucan with various biological activities.

To achieve the above object, the present invention adopts the following technical solutions:

The invention provides a preparation method of high-content Ganoderma lucidum β-glucan and an efficient and rapid detection method thereof. The β-glucan in Ganoderma lucidum is prepared by ethanol-trypsin pretreatment combined with ultrasonic-assisted alkaline hydrothermal extraction, and the prepared β-glucan is purified by DEAE Sephadex A-25 gel column chromatography, and the content of β-glucan was determined by high pressure-acid enzymatic hydrolysis-HPLC method. After methodological investigation, it is found that the method has high detection sensitivity, good stability, good reproducibility and high detection accuracy.

The specific preparation method is as follows:
S1. Ethanol-trypsin pretreatment of Ganoderma lucidum fruiting bodies
weighing 10 g of Ganoderma lucidum fruiting bodies, smashing through a 50-mesh sieve, placing in a stoppered test tube, adding 15 mL of ethanol of a certain concentration, and mixing on a vortex mixer for 20 min, adding 1 g of trypsin and performing enzymatic hydrolysis for 0.5-2 h, to remove impurities such as oil, pigment, and protein in the fruiting bodies, centrifuging at 5000 r/min for 20 min, filtering and discarding the filtrate to obtain filter residue of Ganoderma lucidum fruiting body.
S2. Alkaline hydrothermal extraction of Ganoderma lucidum crude polysaccharide
placing the filter residue of Ganoderma lucidum fruiting body in a 100 mL round-bottomed flask, adding 20 mL of 0.1 mol/mL NaOH solution, and performing ultrasonic treatment in an ultrasonic cleaner at a frequency of 380 W at 60-80° C. for 40 min; after ultrasonication, centrifuging at 5000 r/min for 20 min, filtering, and repeating the above steps for the resulting filter residue, performing hot-extraction with alkaline water once, centrifuging, filtering, and combining the filtrate from two extractions; adjusting pH to neutrality with hydrochloric acid solution of 0.2 mol/mL, performing vacuum concentration on the resulting neutral extraction solution in a rotary evaporator, wherein the neutral extraction solution is concentrated to ¼ of the original volume;
S3. obtaining the crude extract of Ganoderma lucidum β-glucan by alcohol precipitation
adding 2-5 times the volume of 90% ethanol to the extraction solution obtained in S2, and placing it at room temperature for 8-12 hours for alcohol precipitation, centrifuging at 5000 r/min for 20 min, filtering, discarding the supernatant to collect the precipitate, and freeze-drying to obtain the crude extract of Ganoderma lucidum β-glucan;
S4. purification of Ganoderma lucidum β-glucan
dissolving the crude extract of Ganoderma lucidum β-glucan in a certain amount of distilled water, centrifuging to get the supernatant, purifying the supernatant by DEAE Sephadex A-25 glucose gel column chromatography using 0.1 mol/L NaOH solution as a mobile phase, collecting fractions containing β-glucan, concentrating and freeze-drying to obtain high content and high purity Ganoderma β-glucan.

The detection method is as follows:
S1. Preparation of standard solution
accurately weighing a glucose standard, and adding high-purity water to make a standard stock solution of 500 μg/mL;
S2. Preparation of the test solution
accurately weighing 1.0000 g of Ganoderma lucidum β-glucan prepared above, putting in a 50 mL test tube with stopper, adding a certain amount of hydrochloric acid, and mixing on a vortex mixer; placing the test tube in a constant temperature water bath at 40° C. for 20-40 min, and mixing in a mixer for 15 s every 10 min to ensure that all β-glucan is dissolved; transferring the mixed solution to a 100 mL Schott bottle, adding high-purity water to 40 mL and shaking evenly, and placing the Schott bottle in an autoclave at 121° C. for hydrolysis for 60 min; after hydrolysis, cooling to room temperature, adjusting pH to 6-7 with 2 mol/L KOH solution; transferring the resulting hydrolysate to a 50 mL volumetric flask, washing the test tube with 0.2 mol/L pH 5.2 sodium acetate buffer, combining the washing solution in the volumetric flask to make up to volume, and filtering; taking 0.1 mL of the filtrate, adding a certain amount of solution containing β-(1,3)-exo-glucanase and β-glucosidase diluted with 0.2 mol/L pH 5.2 sodium acetate buffer, mixing evenly on a mixer and reacting at 40° C. for 1-2 h, transferring the obtained enzymatic hydrolyzate to a 50 mL volumetric flask to make up to constant volume, to obtain the test solution.
S3. Drawing of standard curve
   taking 1.0, 2.0, 4.0, 5.0, and 10.0 mL of the standard stock solution obtained in S1 and placing in a 25 mL volumetric flask respectively, and diluting to the mark with high-purity water to obtain concentrations of 20, 40, 80, 100, and 200 μg/mL; filtering the standard solutions with a 0.45 μm microporous membrane, and accurately injecting 20 μL of the filtrate in turn into the high performance liquid chromatograph to draw the standard curve;
S4. Sample content determination
filtering the test solution in S2 with a 0.45 μm microporous membrane, and then accurately injecting 20 μL of the filtrate into the high performance liquid chromatograph; after elution, calculating the content of β-glucan in Ganoderma lucidum according to the concentration-peak area standard curve and extraction ratio.

Methodological Investigation

(1) Precision Test

According to the above method, a glucose standard solution with a concentration of 60 μg/mL is prepared. Under the same conditions, the glucose standard solution of known concentration was injected repeatedly for 6 times, the retention time and peak area were measured, and the RSD value (should be less than 3%) was calculated to examine the precision of the instrument.

(2) Repeatability Test

The test solution obtained above was injected repeatedly 6 times under the same conditions, the retention time and peak area were measured, and the RSD value (should be less than 3%) was calculated to examine the repeatability of the instrument.

(3) Stability Test

The test solution prepared above was placed for 0, 2, 4, 8, 12 h and 24 h respectively and injected according to the above chromatographic conditions to check the stability of the sample.

(4) Recovery Test

0.16 mL, 0.20 mL, and 0.24 mL of the standard stock solution are taken, respectively, and placed in a 2 mL volumetric flask and made up to the constant volume with high-purity water, to obtain three glucose standard solutions with concentrations of 40 μg/mL, 50 μg/mL, and 60 μg/mL, respectively. 3 glucose standard solutions of different concentrations are added to the test solution of known concentration respectively, and under the same conditions, the samples are injected and measured, and each concentration is measured 3 times in parallel.

### BRIEF DESCRIPTION OF THE DRAWINGS

FIG. 1 is the chromatogram of reference substance glucose.

FIG. 2 is a graph of the standard curve of glucose.

FIG. 3 is the chromatogram of Ganoderma lucidum β-glucan sample according to embodiment 1.

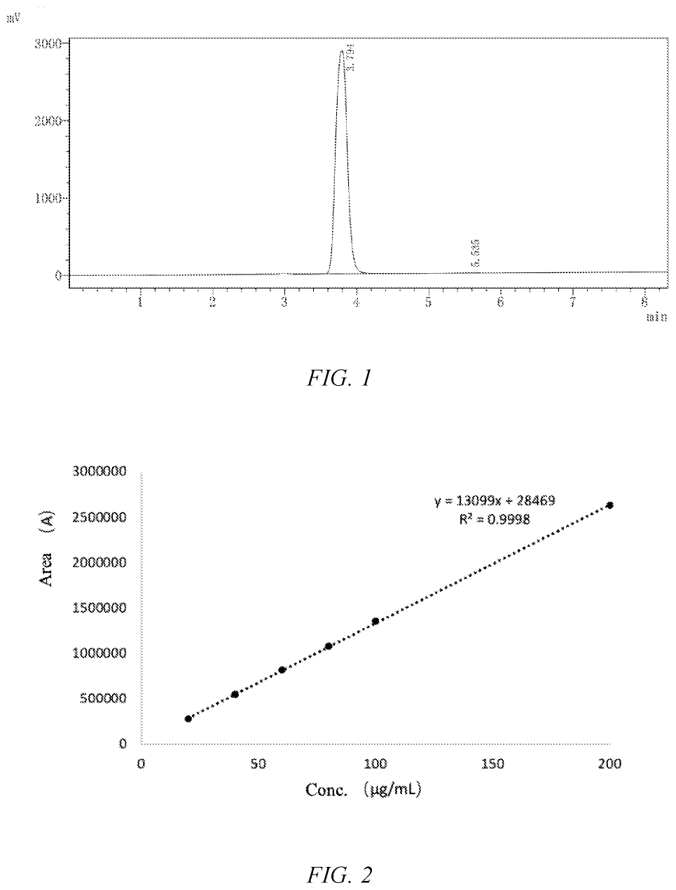

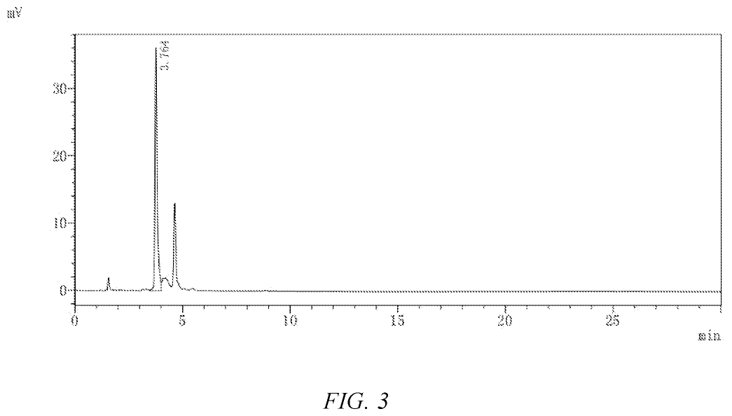

### DETAILED DESCRIPTION OF THE PREFERRED EMBODIMENTS

The instruments used in the present invention are as follows:

Shimadzu-LabSolutions (Shimadzu LC-16, Japan); RID-20A Refractive Index Detector (Shimadzu, Japan); BSA224S 1/10,000 Electronic Balance (Sartorius (Shanghai) Trading Co., Ltd.); ES1035A 1/100,000 electronic balance (Tianjin Deant Sensing Technology Co., Ltd.); GL2202-1SCN 1 percent electronic balance (Sartoris (Shanghai) Trading Co., Ltd.); KQ-300DE Ultrasonic Cleaner (Kunshan Ultrasonic Instrument Co., Ltd.); RV-S Rotary Evaporator (Wuxi Xinghaiwang Biochemical Equipment Co., Ltd.); JC-XW-I Vortex Mixer (Qingdao Juchuang Century Environmental Protection Technology Co., Ltd.); LS-75HD Autoclave (Jiangyin Binjiang); YB-2000A Crusher (Yongkang Sufeng Industry and Trade Co., Ltd.); SJIA-ION Freeze Dryer (Ningbo Shuangjia Instrument Co., Ltd.).

The reagents and materials used in the present invention are as follows:

Food grade pancreatin (purity 99%, Zhejiang Fuxuan Biotechnology Co., Ltd.); DEAE-Sephadex A-25 (purity 99%, Shanghai Zeye Biotechnology Co., Ltd.); glucose standard (purity HPLC≥99.6%, China Institute of Metrology); Ganoderma lucidum polysaccharide (purity GC≥95%, China Institute of Metrology); high-purity water: laboratory-made; sodium acetate buffer (3 mol/L, pH5.2, sterile, Regen Bio); β-(1,3)-Exo-glucanase and β-glucosidase (BR, Spectra (Shanghai) Biotechnology Co., Ltd.).

### Example 1

The specific steps of the preparation method of Ganoderma lucidum β-glucan are as follows:

1. Ethanol-Trypsin Pretreatment of Ganoderma lucidum Fruiting Bodies

10 g of Ganoderma lucidum fruiting bodies was weighed, crushed through a 50-mesh sieve, placed in a test tube with a stopper. 15 mL of ethanol of a certain concentration was added, the resulting mixture was mixed on a vortex mixer for 20 min, then 1 g of trypsin was added for enzymolysis for 0.5-2 h to remove impurities such as oil, pigment, and protein in the fruiting bodies. The solution was centrifuged at 5000 r/min for 20 min, filtered to obtain filter residue of Ganoderma lucidum fruiting bodies after discarding the filtrate.

2. Alkaline Hydrothermal Extraction of Crude Polysaccharide from Ganoderma lucidum

The filter residue of Ganoderma lucidum fruiting bodies was placed in a 100 mL round-bottomed flask, 20 mL of NaOH solution (0.1 mol/mL) was added, and ultrasonic treatment was performed in an ultrasonic cleaner with a frequency of 380 W at 60-80° C. for 40 min. After ultrasonication, the solution was centrifuged at 5000 r/min for 20 min, and filtered. The obtained filter residue was treated by the above steps, and then hot-extracted with alkaline water once, centrifuged, filtered, and the filtrate obtained from two extractions was combined. The pH was adjusted to neutrality with hydrochloric acid solution (0.2 mol/mL). The obtained neutral extract was placed in a rotary evaporator and concentrated under reduced pressure to ¼ of the original volume.

3. Obtaining the Crude Extract of Ganoderma lucidum β-Glucan by Alcohol Precipitation

2-5 times the volume of 90% ethanol was added to the extraction solution obtained in step 2, the resulting solution was placed at room temperature for 8-12 h for alcohol precipitation, centrifuged at 5000 r/min for 20 min, filtered, the precipitate was collected after discarding the supernatant for freeze-drying to obtain the crude extract of Ganoderma lucidum β-glucan.

4. Purification of Ganoderma lucidum β-Glucan

The above solid was dissolved in a certain amount of distilled water, centrifuged to get the supernatant. The supernatant was purified by DEAE Sephadex A-25 Sephadex column chromatography using 0.1 mol/L NaOH solution as the mobile phase, and fractions containing β-glucan were collected, concentrated and freeze-dried to obtain high-content and high-purity Ganoderma β-glucan.

The specific steps of the method for detecting Ganoderma lucidum β-glucan are as follows:

1. Preparation of Standard Solution

The glucose standard was accurately weighed, and high-purity water was added to make a solution of 500 μg/mL to obtain the standard stock solution.

2. Preparation of the Test Solution

1.0000 g of Ganoderma lucidum β-glucan was accurately weighed, placed in a 50 mL test tube with a stopper, a certain amount of hydrochloric acid was added, and mixed on a vortex mixer. The test tube was placed in a constant temperature water bath at 40° C. for 20-40 min, and mixed in a mixer for 15 s every 10 min to ensure that all β-glucan was dissolved. Then, the mixed sample was transferred to a 100 mL Schott bottle, high-purity water was added to 40 mL, mixed uniformly by shaking, and the Schott bottle was placed in an autoclave at 121° C. for hydrolysis for 60 min. After hydrolysis, the hydrolysate was cooled to room temperature, KOH solution (2 mol/L) was added to adjust the pH value to pH 6-7.

The obtained high-pressure hydrolysate was transferred to a 50 mL volumetric flask, the test tube was washed with 0.2 mol/L pH 5.0 sodium acetate buffer, and the washing solutions were combined in the volumetric flask, made up to volume, and filtered. 0.1 mL of the filtrate was taken and a certain amount of solution containing β-(1,3)-exo-glucanase and β-glucosidase diluted with 0.2 mol/L pH 5.0 sodium acetate buffer was added, the resulting solution was mixed evenly on a mixer, reacted at 40° C. for 1-2 h, the obtained enzymatic hydrolyzate was transferred to a 50 mL volumetric flask to make up to constant volume, and the test solution was obtained.

3. Plotting of the Standard Curve

1.0, 2.0, 4.0, 5.0, and 10.0 mL of the standard stock solution obtained in step 1 was taken and placed in a 25 mL volumetric flask respectively, and high-purity water was added to make up the volume to the mark to obtain standard solutions with concentrations of 20, 40, 80, 100, and 200 μg/mL. The standard solution was filtered through a 0.45 μm microporous membrane, and then 20 μL of the filtrate was accurately drawn and injected into the high-performance liquid chromatograph.

TABLE 1
Concentration peak area of glucose standard

| Number | Concentration (μg/mL) | Peak area(A) |
|---|---|---|
| 1 | 20 | 290449 |
| 2 | 40 | 552429 |
| 3 | 80 | 1076389 |
| 4 | 100 | 1338369 |
| 5 | 200 | 2648269 |

According to Table 1, taking the concentration as the abscissa X and the peak area as the ordinate Y, the standard curve was obtained, as shown in FIG. 2. Its glucose standard curve equation is Y=13099X+28469, and its correlation coefficient R²=0.9998, which has a good linear relationship.

4. Determination of Sample Content

The test solution of the step 2 was filtered with a 0.45 μm microporous membrane, and then 20 μL of the filtrate was accurately drawn and injected into a high-performance liquid chromatograph for elution, and chromatogram 3 was obtained. According to the concentration-peak area standard curve, the content of β-glucan in 1 g of the sample was calculated to be 0.918 g, and the calculated extraction yield was 91.8%.

Methodological Investigation:

(1) Precision Test

According to the above method, a glucose standard solution with a concentration of 60 μg/mL was prepared. Under the same conditions, the known concentration of glucose standard solution was repeatedly injected 6 times, the retention time and peak area were measured and the RSD value was calculated, see Table 2. The calculated RSD values are all less than 3%, and the results show that the equipment has good parallel injection precision.

TABLE 2
Precision study of retention times and peak areas

| Injection number | Retention time (min) | Peak area (A) |
|---|---|---|
| 1 | 3.792 | 32032860 |
| 2 | 3.774 | 32433800 |
| 3 | 3.750 | 31007560 |
| 4 | 3.803 | 32100073 |
| 5 | 3.687 | 31870997 |
| 6 | 3.787 | 32692400 |
| RSD (%) | 1.130 | 1.809 |

(2) Repeatability Test

The test solution obtained by the above process was injected 6 times under the same conditions, and the measured retention time and peak area were analyzed and the RSD value was calculated, as shown in Table 3. The calculated RSD values were all less than 3%, and the results showed that the method had good reproducibility.

TABLE 3
Repeatability studies of retention time and peak areas

| Injection number | Retention time (min) | Peak area (A) |
|---|---|---|
| 1 | 3.764 | 262014 |
| 2 | 3.737 | 266138 |
| 3 | 3.627 | 263919 |
| 4 | 3.720 | 265977 |
| 5 | 3.768 | 260020 |
| 6 | 3.744 | 260015 |
| RSD (%) | 1.394 | 1.053 |

(3) Stability Test

The test solution prepared in the above process was placed for 0, 2, 4, 8, 12 h and 24 h and injected according to the above chromatographic conditions. The measured retention time and peak area were analyzed and the RSD value was calculated, see Table 4. The RSD values were all less than 3%, indicating that the sample solution of Ganoderma lucidum polysaccharide has good stability within 24 h at room temperature.

TABLE 4
Stability study of retention time and peak areas

| Time(h) | Retention time(min) | Peak area(A) |
|---|---|---|
| 0 | 3.726 | 229092 |
| 2 | 3.795 | 229034 |
| 4 | 3.747 | 227433 |
| 8 | 3.796 | 223026 |
| 12 | 3.682 | 222965 |
| 24 | 3.710 | 221430 |
| RSD (%) | 1.232 | 1.514 |

(4) Recovery Test

0.16 mL, 0.2 mL, and 0.24 mL of the standard stock solution was taken and placed in a 2 mL volumetric flask, high-purity water was used to make up the volume to obtain three glucose standard solutions with concentrations of 40 μg/mL, 50 μg/mL, and 60 μg/mL, respectively. 3 standard solutions of glucose with different concentrations were added to the test solution of known concentration respectively, under the same conditions, the solution was injected and measured, with 3 times for each concentration in parallel. The measured retention times and peak areas were analyzed and RSD values were calculated. The results are shown in Table 5. The calculated RSD values were all less than 3%. The recovery rate of sample addition was between 98.7% and 104.525%, indicating that the method was accurate.

TABLE 5
Sample recovery rate

| | Concentration | Addition | Measured | | | |
|---|---|---|---|---|---|---|
| | of test | amount | amount | Recovery | Average | |
| Number | solution(μg/mL) | (μg/mL) | (μg) | (%) | recovery(%) | RSD(%) |
| 1 | 50 | 40 | 89.784 | 99.460 | | |
| 2 | 50 | 40 | 91.810 | 104.525 | | |
| 3 | 50 | 40 | 90.414 | 101.036 | | |
| 4 | 50 | 50 | 100.630 | 101.260 | | |
| 5 | 50 | 50 | 101.278 | 102.556 | 100.897 | 1.761 |
| 6 | 50 | 50 | 99.350 | 98.700 | | |
| 7 | 50 | 60 | 110.152 | 100.245 | | |
| 8 | 50 | 60 | 110.454 | 100.757 | | |
| 9 | 50 | 60 | 109.722 | 99.537 | | |

### Example 2

The optimization and comparison of the pretreatment conditions of Ganoderma lucidum fruiting bodies were carried out according to the following methods.

1. Ethanol-Trypsin Pretreatment of Ganoderma lucidum Fruiting Bodies

10 g of Ganoderma lucidum fruiting body was weighed, crushed through a 50-mesh sieve, placed in a test tube with a stopper, 15 mL of ethanol, methanol, and DMF (N,N-dimethylformamide) with a certain concentration was added respectively, and mixed in a vortex for 20 min, then 1 g of trypsin was added for enzymolysis for 0.5-2 h to remove impurities such as oil, pigment, and protein in the fruiting bodies. The resulting solution was centrifuged at 5000 r/min for 20 min, filtered, to obtain the filter residue of Ganoderma lucidum fruiting body after discarding the filtrate.

2. Alkaline Hydrothermal Extraction of Crude Polysaccharide from Ganoderma lucidum

The preparation method of Ganoderma lucidum crude polysaccharide by alkaline hydrothermal extraction is the same as the above-mentioned specific steps.

3. The Crude Extract of Ganoderma lucidum β-Glucan Obtained by Alcohol Precipitation

The operation method for obtaining the crude extract of Ganoderma lucidum β-glucan by alcohol precipitation is the same as the above-mentioned specific steps.

4. Purification of Ganoderma lucidum β-Glucan

The purification method of Ganoderma lucidum β-glucan is the same as the above-mentioned specific steps.

5. Preparation of the Test Solution

The purified β-glucan was treated according to the above specific steps to obtain 3 test solutions.

6. Determination of Sample Content

The three test solutions in step 5 were filtered with a 0.45 μm microporous membrane respectively, and then 20 μL of the filtrate was accurately drawn and injected into a high performance liquid chromatograph for elution. According to the concentration-peak area standard curve, the obtained peak area is converted into concentration, and the results are shown in Table 6:

TABLE 6
Optimization of pretreatment conditions
for Ganoderma lucidum fruiting bodies

| Solvent | Content (g/g) | Extraction ratio (%) |
|---|---|---|
| Ethanol | 0.918 | 91.8 |
| Methanol | 0.877 | 87.7 |
| DMF | 0.895 | 89.5 |

The results show that using ethanol combined with trypsin to pretreat Ganoderma lucidum fruiting bodies to extract Ganoderma β-glucan is more efficient. Therefore, ethanol was selected as the pretreatment reagent.

### Example 3

The comparison of extraction temperature in alkaline hydrothermal extraction of Ganoderma lucidum crude polysaccharide was carried out according to the following method.

1. Ethanol-Trypsin Pretreatment of Ganoderma lucidum Fruiting Bodies

The method of ethanol-trypsin pretreatment of Ganoderma lucidum fruiting bodies is the same as the above-mentioned specific steps.

2. Alkaline Hydrothermal Extraction of Crude Polysaccharide from Ganoderma lucidum

The filter residue of Ganoderma lucidum fruiting body was placed in a 100 mL round-bottomed flask, 20 mL of NaOH solution (0.1 mol/mL) was added, and ultrasonic treatment was performed in an ultrasonic cleaner for 40 min, the frequency was 380 W, the temperatures were 60° C., 70° C., 80° C. respectively. After ultrasonication, the solution was centrifuged at 5000 r/min for 20 min, and filtered. The obtained filter residue was treated by the above steps, and then hot-extracted with alkaline water once, centrifuged, filtered, and the filtrate from two extractions was combined. The pH was adjusted to neutrality with hydrochloric acid solution (0.2 mol/mL). The obtained neutral extract was placed in a rotary evaporator and concentrated under reduced pressure to ¼ of the original volume.

3. The Crude Extract of Ganoderma lucidum β-Glucan Obtained by Alcohol Precipitation

The method for obtaining the crude extract of Ganoderma lucidum β-glucan by alcohol precipitation is the same as the above-mentioned specific steps.

4. Purification of Ganoderma lucidum β-Glucan

The purification method of Ganoderma lucidum β-glucan is the same as the above-mentioned specific steps.

5. Preparation of the Test Solution

The purified β-glucan was processed according to the above specific steps to obtain 3 test solutions.

6. Determination of Sample Content

The three test solutions in step 5 were filtered with a 0.45 μm microporous membrane respectively, and then 20 μL of the filtrate was accurately drawn and injected into a high performance liquid chromatograph for elution. According to the concentration-peak area standard curve, the obtained peak area was converted into concentration, and the results are shown in Table 7.

TABLE 7
Contents of β-glucan extracted from
Ganoderma lucidum at different temperatures

| Ultrasonication temperature (° C.) | Content (g/g) | Extraction ratio (%) |
|---|---|---|
| 60 | 0.882 | 88.2 |
| 70 | 0.918 | 91.8 |
| 80 | 0.912 | 91.2 |

The results shows that when the ultrasonic temperature used in the ultrasonic-assisted alkaline hydrothermal extraction of Ganoderma lucidum polysaccharide was 70° C., the preparation efficiency of Ganoderma lucidum β-glucan was higher. Therefore, 70° C. was chosen as the ultrasonic temperature.

### Example 4

The comparison of different concentrations of alkaline water in the thermal extraction of Ganoderma lucidum crude polysaccharide is carried out according to the following method.

1. Ethanol-Trypsin Pretreatment of Ganoderma lucidum Fruiting Bodies

The method of ethanol-trypsin pretreatment of Ganoderma lucidum fruiting bodies is the same as the above-mentioned specific steps.

2. Alkaline Hydrothermal Extraction of Crude Polysaccharide from Ganoderma lucidum

The filter residue of Ganoderma lucidum fruiting body was placed in a 100 mL round-bottomed flask, and 20 mL of 0.01 mol/mL NaOH solution, 0.1 mol/mL NaOH solution, and 1 mol/mL NaOH solution were added respectively and placed in an ultrasonic cleaner for ultrasonic treatment for 40 min, the frequency was 380 W, and the temperature was 60-80° C. After ultrasonication, the solution was centrifuged at 5000 r/min for 20 min, and filtered. The obtained filter residue was treated by the above steps, and hot-extracted with alkaline water once, centrifuged, filtered, and the filtrate from two extractions was combined. The pH was adjusted to neutrality with hydrochloric acid solution (0.2 mol/mL). The obtained neutral extraction solution was placed in a rotary evaporator and concentrated under reduced pressure to ¼ of the original volume.

3. The Crude Extract of Ganoderma lucidum β-Glucan Obtained by Alcohol Precipitation

The method for obtaining the crude extract of Ganoderma lucidum β-glucan by alcohol precipitation is the same as the above-mentioned specific steps.

4. Purification of Ganoderma lucidum β-Glucan

The purification method of Ganoderma lucidum β-glucan is the same as the above-mentioned specific steps.

5. Preparation of the Test Solution

The purified β-glucan was processed according to the above specific steps to obtain 3 test solutions.

6. Determination of Sample Content

The three test solutions in step 5 were filtered with a 0.45 μm microporous membrane respectively, and then 20 μL of the filtrate was accurately drawn and injected into a high performance liquid chromatograph for elution. According to the concentration-peak area standard curve, the obtained peak area is converted into concentration, and the results are shown in Table 8:

TABLE 8
Contents of β-glucan extracted from Ganoderma lucidum
with different concentrations of alkaline water

| NaOH concentration (mol/mL) | Content (g/g) | Extraction ratio (%) |
|---|---|---|
| 0.01 | 0.853 | 85.3 |
| 0.1 | 0.918 | 91.8 |
| 1 | 0.890 | 89.0 |

The results shows that when the concentration of NaOH used in ultrasonic-assisted alkaline hydrothermal extraction of Ganoderma lucidum crude polysaccharide was 0.1 mol/mL, the preparation efficiency of Ganoderma lucidum β-glucan was higher. Therefore, 0.1 mol/mL NaOH was selected for the extraction.

The above content is only to illustrate the technical idea of the present invention, and cannot limit the protection scope of the present invention. Any modification made on the basis of the technical solution proposed in accordance with the technical idea of the present invention falls within the scope of the claims of the present invention.

What is claimed is:

1. A preparation method of high-content Ganoderma lucidum β-glucan and an efficient and rapid detection method thereof, the preparation method comprising ethanol-trypsin pretreatment and ultrasonic-assisted alkaline hydrothermal extraction of Ganoderma lucidum β-glucan by using Ganoderma lucidum fruiting body as a raw material, and purifying the β-glucan by DEAE Sephadex A-25 Sephadex column chromatography, and the detection method comprising quantitatively detecting and analyzing the content of the prepared β-glucan by high pressure-acid enzymatic hydrolysis-HPLC method, wherein:
the preparation method of high-content Ganoderma lucidum β-glucan comprises:
S1, ethanol-trypsin pretreatment of Ganoderma lucidum fruiting bodies: weighing 10 g of Ganoderma lucidum fruiting bodies, smashing through a 50-mesh sieve, placing in a stoppered test tube, adding 15 mL of ethanol of a certain concentration, and mixing on a vortex mixer for 20 min, adding 1 g of trypsin and performing enzymatic hydrolysis for 0.5-2 h, to remove impurities such as oil, pigment, and protein in the fruiting bodies, centrifuging at 5000 r/min for 20 min, filtering and discarding the filtrate to obtain filter residue of Ganoderma lucidum fruiting body;
S2, ultrasound-assisted alkaline hydrothermal extraction of Ganoderma lucidum crude polysaccharide: placing the filter residue of Ganoderma lucidum fruiting body in a 100 mL round-bottomed flask, adding 20 mL of 0.1 mol/mL NaOH solution, and performing ultrasonic treatment in an ultrasonic cleaner at a frequency of 380 W at 60-80° C. for 40 min; after ultrasonication, centrifuging at 5000 r/min for 20 min, filtering, and repeating the above steps for the resulting filter residue, performing hot-extraction with alkaline water once, centrifuging, filtering, and combining the filtrate from two extractions; adjusting pH to neutrality with hydrochloric acid solution of 0.2 mol/mL, performing vacuum concentration on the resulting neutral extraction solution in a rotary evaporator, wherein the neutral extraction solution is concentrated to ¼ of the original volume;
S3, obtaining the crude extract of Ganoderma lucidum β-glucan by alcohol precipitation: adding 2-5 times the volume of 90% ethanol to the extraction solution obtained in S2, and placing it at room temperature for 8-12 hours for alcohol precipitation, centrifuging at 5000 r/min for 20 min, filtering, discarding the supernatant to collect the precipitate, and freeze-drying to obtain the crude extract of Ganoderma lucidum β-glucan;
S4, purification of Ganoderma lucidum β-glucan: dissolving the crude extract of Ganoderma lucidum β-glucan in a certain amount of distilled water, centrifuging to get the supernatant, purifying the supernatant by DEAE Sephadex A-25 glucose gel column chromatography using 0.1 mol/L NaOH solution as a mobile phase, collecting fractions containing β-glucan, concentrating and freeze-drying to obtain high content and high purity Ganoderma β-glucan;
the efficient and rapid detection method of the high-content Ganoderma lucidum β-glucan comprises:
S1, preparation of a standard solution
accurately weighing a glucose standard, and adding high-purity water to make a standard stock solution of 500 μg/mL;
S2, preparation of a test solution
accurately weighing 1.0000 g of Ganoderma lucidum β-glucan prepared above, putting in a 50 mL test tube with stopper, adding a certain amount of hydrochloric acid, and mixing on a vortex mixer; placing the test tube in a constant temperature water bath at 40° C. for 20-40 min, and mixing in a mixer for 15 s every 10 min to ensure that all β-glucan is dissolved; transferring the mixed solution to a 100 mL Schott bottle, adding high-purity water to 40 mL and shaking evenly, and placing the Schott bottle in an autoclave at 121° C. for hydrolysis for 60 min; after hydrolysis, cooling to room temperature, adjusting pH to 6-7 with 2 mol/L KOH solution;
transferring the resulting hydrolysate to a 50 mL volumetric flask, washing the test tube with 0.2 mol/L pH 5.2 sodium acetate buffer, combining the washing solution in the volumetric flask to make up to volume, and filtering; taking 0.1 mL of the filtrate, adding a certain amount of solution containing β-(1,3)-exo-glucanase and β-glucosidase diluted with 0.2 mol/L pH 5.2 sodium acetate buffer, mixing evenly on a mixer and reacting at 40° C. for 1-2 h, transferring the obtained enzymatic hydrolyzate to a 50 mL volumetric flask to make up to constant volume, to obtain the test solution;
S3, drawing of standard curve
taking 1.0, 2.0, 4.0, 5.0, and 10.0 mL of the standard stock solution obtained in S1 and placing in a 25 mL volumetric flask respectively, and diluting to the mark with high-purity water to obtain concentrations of 20, 40, 80, 100, and 200 μg/mL; filtering the standard solutions with a 0.45 μm microporous membrane, and accurately injecting 20 μL of the filtrate in turn into the high performance liquid chromatograph to draw the standard curve;
S4, sample content determination
filtering the test solution in S2 with a 0.45 μm microporous membrane, and then accurately injecting 20 μL of the filtrate into the high performance liquid chromatograph; after elution, calculating the content of β-glucan in Ganoderma lucidum according to the concentration-peak area standard curve and extraction ratio.

2. The preparation method of high-content Ganoderma lucidum β-glucan according to claim 1, wherein Ganoderma lucidum fruiting body is used as a raw material.

3. The preparation method of high-content Ganoderma lucidum β-glucan according to claim 1, wherein in S1, 15 mL of 60% ethanol is added.

4. The preparation method of high-content Ganoderma lucidum β-glucan according to claim 1, wherein in S1, 1 mL of trypsin is added, and enzymatic hydrolysis is carried out for 1 h.

5. The preparation method of high-content Ganoderma lucidum β-glucan according to claim 1, wherein the ultrasonic treatment is performed at 70° C. in S2.

6. The preparation method of high-content Ganoderma lucidum β-glucan according to claim 1, wherein: 4 times the volume of 90% ethanol is added, and the resulting solution is placed at room temperature for 12 h for alcohol precipitation.

7. The efficient and rapid detection method of high-content Ganoderma lucidum β-glucan according to claim 1, wherein in S2, 1 mL hydrochloric acid of 2 mol/mL is accurately added.

8. The efficient and rapid detection method of high-content Ganoderma lucidum β-glucan according to claim 1, wherein in S2 the test tube is placed in a constant temperature water bath for 30 min.

9. The efficient and rapid detection method of high-content Ganoderma lucidum β-glucan according to claim 1, wherein in S2, 0.1 mL of solution containing β-(1,3)-Exo-glucanase and β-glucosidase diluted with 0.2 mol/L pH 5.2 sodium acetate buffer is added.

10. The efficient and rapid detection method of high-content Ganoderma lucidum β-glucan according to claim 1, wherein in S2 after mixing the reaction is performed at 40° C. for 1 h.

11. The efficient and rapid detection method of high-content Ganoderma lucidum β-glucan according to claim 1, wherein in S2 and S3 the high-performance liquid chromatography is performed with:
chromatographic column: Shodex SH1011 column (8.0 mm×300 mm, 6 μm),
mobile phase: high purity water,
flow rate: 0.5 mL/min,
column temperature: 80° C.,
refractive index detector temperature: 35° C., and
injection volume: 20 μL.
